(19) United States
(12) Patent Application Publication
(10) Pub. No.: US 2005/0169865 A1
(43) Pub. Date: Aug. 4, 2005

(54) Cosmetic composition comprising at least one cationic agent, at least one semi-crystalline polymer and at least one oil, and cosmetic treatment process

(76) Inventor: Eric Parris, Clichy (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/014,860

(22) Filed: Dec. 20, 2004

Related U.S. Application Data
(60) Provisional application No. 60/532,891, filed on Dec. 30, 2003.

(30) Foreign Application Priority Data
Dec. 19, 2003 (FR) 03 51144

(51) Int. Cl. A61K 7/06; A61K 7/11

(52) U.S. Cl. 424/70.11

(57) ABSTRACT

The present disclosure relates to a cosmetic composition comprising, in a cosmetically acceptable medium, at least one cationic agent, at least one oil and at least one semi-crystalline polymer having a melting point of greater than or equal to 30° C. The present disclosure further relates and to a process for cosmetic treatment of keratinous substances, such as the hair, with the compositions as disclosed herein.

This application claims benefit of U.S. Provisional Application No. 60/532,891, filed Dec. 30, 2003.

The present disclosure relates to a cosmetic composition, for instance, for conditioning the hair, comprising at least one cationic agent and at least one semi-crystalline polymer in combination with at least one oil, and to a process for the cosmetic treatment of keratinous substances, such as the hair.

It is known that hair that has been sensitized (i.e., damaged and/or embrittled) to various extents under the action of atmospheric agents or under the action of mechanical or chemical treatments, such as dyeings, bleachings and/or permings, often can be difficult to disentangle and to style, and can lack softness.

In the field of cosmetics, one aim, for example, is to improve the conditioning of the hair. The term “conditioning” can be understood to mean properties of easy disentangling, of sheen, of softness to the touch and of sleekness.

Cosmetic compositions comprising cationic surfactants have already been provided for the treatment of keratinous substances and for instance, the hair. However, there can be disadvantages to such compositions, such as problems of rinsability, problems of stability, difficulties of distribution over keratinous substances, and inadequate cosmetic properties.

The use of cationic polymers, of cationic silicones or of cationic surfactants has been recommended, in compositions for washing or caring for keratinous substances, such as the hair, in order to facilitate the disentangling of the hair and to provide it with softness and suppleness. There can be various disadvantages to the use of cationic polymers or cations for this purpose. Because of their high affinity for the hair, some of these polymers are deposited to a significant extent when repeatedly used and can result in undesirable effects, such as an unpleasant heavy feel, stiffening of the hair and an interfiber adhesion which can affect the styling.

In summary, current conditioning cosmetic compositions are not completely satisfactory. Thus, there is a need to obtain cosmetic compositions having improved conditioning properties, for example, a smoother feel.

The use of semi-crystalline polymers is known in cosmetics, and for instance, in the field of makeup, as is disclosed in French Patent Application No. FR 2,824,267. Cosmetic compositions, the fatty phase(s) of which are gelled by semi-crystalline polymers, have been disclosed in French Patent Application No. FR 2,824,264, which discloses, for instance, solid lipstick compositions in the form of a stick. U.S. Pat. Nos. 5,736,125 and 5,156,911, and PCT Application No. WO 01/19333, illustrate, in a non-limiting manner, certain types of semi-crystalline polymers which can participate in the composition of the formulations of the present disclosure. However, these documents do not disclose cosmetic compositions comprising a cationic surfactant.

It has been discovered by the present inventors that the combination of at least one cationic agent, at least one semi-crystalline polymer and at least one oil, for instance in a nondetergent media having a low concentration (such as less than 2% by weight with respect to the total weight of the composition) or zero concentration of washing surface-active agents, can make it possible to overcome at least one of these disadvantages.

The hair treated with this composition may can be smooth, glossy, and/or soft, and/or may disentangle easily, have separate individual hairs and/or have a soft and residue-free feel. The hair may also have a natural and non-heavy appearance.

Without being committed to any one theory, it would appear that, under these conditions, the deposition of oil on the hair is significantly increased, resulting in an increased effectiveness. However, this improvement can be made without having a heavy greasy feel, which is usually the case when the amount of oil is increased.

Furthermore, this conditioning effect can be persistent with regard to rinsing.

Accordingly, the present disclosure relates to novel, cosmetic compositions, for example, nonwashing compositions, comprising, in a cosmetically acceptable medium, at least one cationic agent, at least one oil and at least one semi-crystalline polymer having a melting point of greater than or equal to 30° C.

Another aspect of the present disclosure comprises a process for the cosmetic treatment of keratinous substances, such as the hair, employing the abovementioned composition.

A further aspect of the present disclosure is the use of the composition as a conditioner.

Other aspects, characteristics, and advantages of the present disclosure will become more apparent upon reading the description and the various examples which follow.

According to one embodiment of the present disclosure, the at least one oil is, for example, pre-thickened by the at least one semi-crystalline polymer, that is to say that the at least one oil and the at least one semi-crystalline polymer are mixed before the introduction into the composition.

The weight ratio of the at least one oil to the at least one polymer as disclosed herein can be, for example, greater than or equal to 50/50, for instance, greater than or equal to 60/40, such as ranging from 60/40 to 99/1, for example ranging from 80/20 to 99/1.

The at least one oil, which may be pre-thickened, is dispersed in the form of particles in the aqueous composition. The oil particles may, for example, exhibit a number-average primary size ranging from 1 μm to 100 μm, for instance ranging from 5 μm to 30 μm, such as ranging from 10 μm to 20 μm.

As used herein, the term “particle primary size” is understood to mean the maximum dimension which it is possible to measure between two diametrically opposite points of an individual particle. The size can be determined, for example, by transmission electron microscopy or from the measurement of the specific surface by the BET method or else via a laser particle sizer.

As used herein, the term “semi-crystalline polymer” is understood to mean polymers comprising a crystallizable part and an amorphous part in the backbone and exhibiting a first-order reversible phase change temperature, for example a melting point (solid-liquid transition). The crystallizable part is either a side chain (or pendent chain) or a block in the backbone.

As used herein, the term “polymers” is understood to mean compounds comprising at least two repeating units, for example, at least three repeating units, such as at least ten repeating units.

When the crystallizable part is a block of the polymer backbone, this crystallizable block has a different chemical nature from that of the amorphous blocks; in this case, the semi-crystalline polymer is a block copolymer, for example of the diblock, triblock or multiblock type. When the crystallizable part is a chain pendent to the backbone, the semi-crystalline polymer can be a homopolymer or a copolymer.

As used herein, the term “crystallizable chain or block” is understood to mean a chain or block which, if it were alone, would change reversibly from the amorphous state to the crystalline state, according to whether the temperature is above or below the melting point. The term “chain,” as used herein, is understood to mean a group of atoms which is in the pendent or side position with respect to the backbone of the polymer. The term “block,” as used herein, is understood to mean a group of atoms belonging to the backbone, a group constituting one of the repeating units of the polymer.

As used herein, the term “organic compound” or “with an organic structure” is understood to mean compounds comprising carbon atoms and hydrogen atoms and optionally heteroatoms, such as S, O, N or P, alone or in combination.

Semi-Crystalline Polymers

The at least one semi-crystalline polymer of the composition of the present disclosure can have, for example, a weight-average molecular mass Mw greater than or equal to 1000, such as ranging from 5,000 to 1,000,000, for instance ranging from 10,000 to 500,000, or from 15,000 to 500,000.

The at least one semi-crystalline polymer comprises, for example, i) a polymer backbone and ii) at least one crystallizable side chain and/or one crystallizable organic block forming part of the backbone of the said semi-crystalline polymer. The at least one semi-crystalline polymer can be chosen for example, from block copolymers comprising at least one crystallizable block and at least one amorphous block, homopolymers and copolymers carrying at least one crystallizable side chain per repeat unit, and the mixtures thereof.

The at least one semi-crystalline polymer according to the present disclosure, acting as structuring agent(s), is solid at ambient temperature (25° C.) and atmospheric pressure (760 mm of Hg), and has a melting point greater than or equal to 30° C., for instance, ranging from 30° C. to 80° C., such as ranging from 30° C. to 70° C. This melting point is a first-order change of state temperature. This melting point can be measured by any known method, for example, using a differential scanning calorimeter (DSC). The melting point values can correspond for example, to the melting point measured using a differential scanning calorimeter (DSC), such as the calorimeter sold under the name DSC 30 by Mettler, with a rise in temperature ranging from 5° C. to 10° C. per minute (the melting point considered is the point corresponding to the temperature of the most endothermic peak of the thermogram).

The at least one semi-crystalline polymer according to the present disclosure can have, for instance, a melting point greater than the temperature of the keratinous substrate intended to receive the composition, such as the hair.

The at least one semi-crystalline polymer as disclosed herein is capable, alone or as a mixture, of structuring (i.e., thickening) the oil without addition of specific surfactant or of salt.

According to the present disclosure, the at least one semi-crystalline polymer can be, for example, soluble in the fatty phase, for instance, to at least 1% by weight, at a temperature greater than the melting point. Apart from the crystallizable chains or blocks, the blocks of the polymers are amorphous.

In one embodiment of the present disclosure, the polymer backbone of the at least one semi-crystalline polymer is soluble in the liquid fatty phase.

For example, the crystallizable blocks or chains of the at least one semi-crystalline polymer can be present in an amount of at least 30% of the total weight of each polymer, such as at least 40%. The at least one semi-crystalline polymer, as disclosed herein, with crystallizable side chains are homo- or copolymers. The at least one semi-crystalline polymer, as disclosed herein, with crystallizable blocks are block or multiblock copolymers. They can be obtained by polymerization of a monomer(s) with reactive double (or ethylenic) bonds or by polycondensation. In one embodiment of the present disclosure, when the at least one crystalline polymer is a polymer with crystallizable side chains, the at least one polymer is in the statistical or random form.

For example, in another embodiment, the at least one semi-crystalline polymer of the present disclosure is synthetic in origin. In yet another embodiment, the at least one semi-crystalline polymer does not comprise a polysaccharide backbone.

Among the semi-crystalline polymers which can be used as disclosed herein, non-limiting mention can be made of, for example:
block copolymers of polyolefins with controlled crystallization, the monomers of which are disclosed in EP-A-0 951 897, polycondensates, for example, of aliphatic or aromatic or aliphatic/aromatic polyester type,
homo- and copolymers carrying at least one crystallizable side chain and homo- and copolymers carrying in the backbone, at least one crystallizable block, such as those disclosed in U.S. Pat. No. 5,156,911,
homo- and copolymers carrying at least one crystallizable side chain, such as with fluorinated group(s), as disclosed in WO-A-01/19333,
and the mixtures thereof. In the last two cases, the crystallizable side chain or block or side chains or blocks are hydrophobic.
   Semi-Crystalline Polymers with Crystallizable Side Chains

Non-limiting mention may be made, for example, of those defined in the U.S. Pat. No. 5,156,911 and WO-A-01/19333. These are homopolymers or copolymers comprising from 50% to 100% by weight of units resulting from the polymerization of at least one monomer carrying a crystallizable hydrophobic side chain.

These homo- and copolymers are of any nature, provided that they exhibit the conditions indicated below, with, for example, the characteristic of being soluble or dispersible in the liquid fatty phase by heating above their melting point M.p. The homo- and copolymers can result:
from the polymerization, such as free-radical polymerization, of at least one monomer with double bond(s) or ethylenic monomers reactive with respect to polymerization, for example, with a vinyl, (meth)acrylic or allyl group;
from the polycondensation of at least one monomer carrying coreactive groups (carboxylic or sulphonic acid, alcohol, amine or isocyanate groups), such as, for example, polyesters, polyurethanes, polyethers, polyureas or polyamides.

Generally, the crystallizable units (chains or blocks) of the semi-crystalline polymers according to the present disclosure originate from monomer(s) with crystallizable block(s) or chain(s) used for the manufacture of the semi-crystalline polymers. These polymers can be chosen, for example, from the homopolymers and copolymers resulting from the polymerization of at least one monomer with crystallizable chain(s) chosen from those of formula (I): 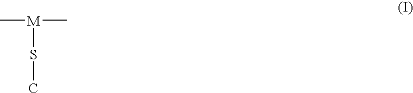
wherein M is an atom of the polymer backbone, S is a spacer and C is a crystallizable group.

The crystallizable chains “—S—C” can be aliphatic or aromatic and optionally fluorinated or perfluorinated. “S” can be chosen from, for example, linear, branched, and cyclic (CH₂)ₙ, (CH₂CH₂O)ₙ, and (CH₂O)ₙ groups, wherein n is an integer ranging from 0 to 22. In one embodiment of the present disclosure, “S” is a linear group. In another embodiment, “S” and “C” are different.

When the crystallizable chains are aliphatic hydrocarbonaceous chains, they comprise hydrocarbonaceous alkyl chains with at least 11 carbon atoms and at most 40 carbon atoms, for example, at most 24 carbon atoms. They can be, for instance, aliphatic chains or alkyl chains having at least 12 carbon atoms, such as C₁₄-C₂₄, for example, C₁₆-C₂₂, alkyl chains. When they are fluorinated or perfluorinated alkyl chains, they comprise at least 11 carbon atoms, at least 6 carbon atoms of which are fluorinated.

Non-limiting mention may be made, as examples of semi-crystalline homopolymers or copolymers with crystallizable chain(s), of those resulting from the polymerization of at least one of the following monomers: saturated alkyl(meth)acrylates with a C₁₄-C₂₄ alkyl group; perfluoroalkyl(meth)acrylates with a C₁₁-C₁₅ perfluoroalkyl group; N-alkyl(meth)acrylamides with a C₁₄ to C₂₄ alkyl group, with or without at least one fluorine atom; vinyl esters with alkyl or perfluoroalkyl chains with a C₁₄ to C₂₄ alkyl group (with at least 6 fluorine atoms per one perfluoroalkyl chain); vinyl ethers with alkyl or perfluoroalkyl chains with a C₁₄ to C₂₄ alkyl group and at least 6 fluorine atoms per one perfluoroalkyl chain; C₁₄ to C₂₄ α-olefins, such as, for example, octadecene, para-alkylstyrenes with an alkyl group comprising from 12 to 24 carbon atoms, and mixtures thereof.

When the polymers result from a polycondensation, the crystallizable hydrocarbonaceous and/or fluorinated chains as defined above are carried via a monomer chosen from diacid, diol, diamine and diisocyanate monomers.

When the polymers as disclosed herein are copolymers, they additionally comprise from 0 to 50% of Y or Z groups resulting from the copolymerization:
of Y, which is chosen from polar and nonpolar monomers, and mixtures thereof; and
of Z, which is chosen from a polar monomer or a mixture of polar monomers.

When Y is a polar monomer, it is chosen from monomers carrying polyoxyalkylenated (such as oxyethylenated and/or oxypropylenated) groups; hydroxyalkyl (meth)acrylates, such as hydroxyethyl acrylate; (meth)acrylamides; N-alkyl(meth)acrylamides; N,N-dialkyl(meth)acrylamides; such as, for example, N,N-diisopropylacrylamide; N-vinylpyrrolidones (NVP); N-vinylcaprolactams; monomers carrying at least one carboxylic acid group, such as (meth)acrylic acid, crotonic acid, itaconic acid, maleic acid and fumaric acid; monomers carrying a carboxylic acid anhydride group, such as maleic anhydride, and mixtures thereof.

When Y is a nonpolar monomer, it is chosen from esters of the linear, branched and cyclic alkyl(meth)acrylate type, vinyl esters, alkyl vinyl ethers, α-olefins, styrenes, styrenes substituted by a C₁ to C₁₀ alkyl group, such as α-methylstyrene, and macromonomers of the polyorganosiloxane with vinyl unsaturation type.

As used herein, the term “alkyl” is understood to mean a saturated group, for example, a saturated C₈ to C₂₄ group, unless specifically mentioned.

As disclosed herein, Z has the same definition as the “polar Y” defined above.

For example, the semi-crystalline polymers with a crystallizable side chain may be chosen from alkyl(meth)acrylate and alkyl(meth)acrylamide homopolymers with an alkyl group as defined above, for instance a C₁₄-C₂₄ alkyl group, copolymers of these monomers with a hydrophilic monomer, for example, different in nature from (meth)acrylic acid, such as N-vinylpyrrolidone and hydroxyethyl(meth)acrylate, and mixtures thereof.

Polymers Carrying at Least one Crystallizable Block in the Backbone

These are again polymers which are soluble or dispersible in the liquid fatty phase by heating above their melting point M.p. These polymers can be, for example, block copolymers composed of at least two blocks of different chemical natures, one of which is crystallizable. For instance, among the polymers that may be used as disclosed herein, non-limiting mention may be made of:
the polymers defined in U.S. Pat. No. 5,156,911
block copolymers of olefin or of cycloolefin with a crystallizable chain, such as those resulting from the block polymerization of:
   cyclobutene, cyclohexene, cyclooctene, norbornene (i.e., bicyclo[2.2.1]hept-2-ene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene and mixtures thereof; with
   ethylene, propylene, 1-butene, 3-methyl-1-butene, 1-hexene, 4-methyl-1-pentene, 1-octene, 1-decene, 1-icosene and mixtures thereof,

For example, in one embodiment of the present disclosure, block copoly(ethylene/norbornene)s and (ethylene/propylene/ethylidenenorbornene) block terpolymers are used.

Use may also be made of polymers resulting from the block copolymerization of at least two C₂-C₁₆ α-olefins, for instance C₂-C₁₂ α-olefins, such as those mentioned above, and for example, the block bipolymers of ethylene and of 1-octene.

The copolymers can be copolymers exhibiting at least one crystallizable block, the remainder of the copolymer being amorphous (at ambient temperature). These copolymers can, in addition, exhibit two crystallizable blocks of different chemical nature. For instance, the copolymers can have, at ambient temperature, both a crystallizable block and a both hydrophobic and lipophilic amorphous block which are sequentially distributed; non-limiting mention may be made, for example, of the polymers having one of the following crystallizable blocks and one of the following amorphous blocks: Blocks crystallizable by nature: polyesters, such as poly(alkylene terephthalate)s, and polyolefins, such as polyethylenes and polypropylenes. Amorphous and lipophilic blocks, such as: amorphous polyolefins and copoly(olefin)s, for example poly(isobutylene), hydrogenated polybutadiene and hydrogenated poly(isoprene).

As examples of such copolymers with a crystallizable block and with an amorphous block, non-limiting mention may be made of:

Poly(ε-caprolactone)-b-poly(butadiene) block copolymers, for example, optionally used hydrogenated, such as those described in the paper, “Melting behavior of poly(ε-caprolactone)-block-polybutadiene copolymers,” by S. Nojima, Macromolecules, 32, 3727-3734 (1999).

Block or multiblock hydrogenated poly(butylene terephthalate)-b-poly(isoprene) block copolymers, cited in the paper, “Study of morphological and mechanical properties of PP/PBT,” by B. Boutevin et al., Polymer Bulletin, 34, 117-123 (1995).

The poly(ethylene)-b-copoly(ethylene/propylene) block copolymers cited in the papers, “Morphology of semi-crystalline block copolymers of ethylene-(ethylene-alt-propylene),” by P. Rangarajan et al., Macromolecules, 26, 4640-4645 (1993) and, “Polymer aggregates with crystalline cores: the system poly(ethylene)-poly(ethylene-propylene),” P. Richter et al., Macromolecules, 30, 1053-1068 (1997).

The poly(ethylene)-b-poly(ethylethylene) block copolymers cited in the general article, “Crystallization in block copolymers,” by I. W. Hamley, Advances in Polymer Science, vol. 148, 113-137 (1999).

The at least one semi-crystalline polymer of the composition as disclosed herein may or may not be crosslinked provided that if there is crosslinking, the degree of crosslinking is not harmful to their dissolution or dispersion in the liquid fatty phase by heating above their melting point. The crosslinking can then be chemical crosslinking, by reaction with a multifunctional monomer during the polymerization. It can also be physical crosslinking, which can then be due either to the establishment of bonds of hydrogen or dipolar type between groups carried by the polymer, such as, for example, dipolar interactions between carboxylate ionomers, these interactions being low in degree and carried by the backbone of the polymer, or to phase separation between the crystallizable blocks and the amorphous blocks carried by the polymer.

According to one aspect of the present disclosure, the at least one semi-crystalline polymer is chosen from the copolymers resulting from the polymerization of at least one monomer with a crystallizable chain, chosen from saturated C₁₄ to C₂₄ alkyl (meth)acrylates; C₁₁ to C₁₅ perfluoroalkyl(meth)acrylates; N—(C₁₄ to C₂₄ alkyl)(meth)acrylamides, with or without at least one fluorine atom; vinyl esters with C₁₄ to C₂₄ alkyl or perfluoroalkyl chains; vinyl ethers with C₁₄ to C₂₄ alkyl or perfluoroalkyl chains; C₁₄ to C₂₄ α-olefins; and para-alkylstyrenes with an alkyl group comprising from 12 to 24 carbon atoms, with at least one optionally fluorinated C₁ to C₁₀ monocarboxylic acid ester or amide, corresponding to those of formula (II): 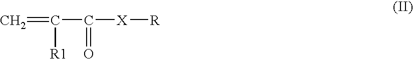
wherein R₁ is chosen from a hydrogen atom and CH₃, R is chosen from optionally fluorinated C₁-C₁₀ alkyl groups and X is chosen from an oxygen atom, and NH and NR₂ groups, wherein R₂ is chosen from optionally fluorinated C₁-C₁₀ alkyl groups.

As specific examples of structuring semi-crystalline polymers which can be used in the composition according to the present disclosure, non-limiting mention may be made of the Intelimer® products from Landec described in the brochure “Intelimer® polymers”, Landec IP22 (Rev. 4-97). These polymers are in the solid form at ambient temperature (25° C.), they carry crystallizable side chains, and exhibit the above formula (I).

With respect to the semi-crystalline polymer generally as disclosed herein, a semi-crystalline polymer having a melting point M.p.₂ of less than 50° C. will be called “polymer with a low melting point” and a crystalline or semi-crystalline polymer having a melting point M.p.₁ of greater than or equal to 50° C. will be called “polymer with a high melting point.” According to the present disclosure, the melting point can be measured for example, by any known method, such as with a differential scanning calorimeter (DSC).

According to the present disclosure, a semi-crystalline polymer with a high melting point can be, for example, chosen from polymers having a melting point M.p.₁ such that 50° C.≦M.p.₁≦150° C., for instance, 55° C.≦M.p.₁≦150° C., such as 60° C.<M.p.₁≦130° C. The semi-crystalline polymers with a low melting point can, for example, have a melting point M.p.₂ such that 30° C.≦M.p.₂<50° C., for instance 35° C.≦M.p.₂≦45° C. This melting point is a first-order change of state temperature.

Generally, the polymers with a low melting point exhibit a melting point M.p.₂ at least equal to the temperature of the keratinous substrate which is to receive the presently disclosed composition.

Non-limiting mention may be made, among semi-crystalline polymers with a high melting point that can be used as disclosed herein, of crystalline polymers that are solid at ambient temperature and that have a melting point of greater than 50° C., such as random polymers comprising controlled crystallization, as disclosed in EP-A-0 951 897, and also, for instance, the commercial products Engage 8 401 and Engage 8 402 from Dupont de Nemours, respectively with melting points of 51° C. and 64° C., which are random ethylene/1-octene bipolymers. These semi-crystalline polymers exhibiting a melting point of greater than or equal to 50° C. include the Intelimer described in the brochure “Intelimer® polymers”, Landec IP22 (Rev. 4-97), with a melting point of 56° C., which is an impermeable and non-sticky product which is viscous at ambient temperature. Use may also be made of the semi-crystalline polymers obtained by copolymerization of behenyl acrylate and of acrylic acid or of NVP, as disclosed in the U.S. Pat. No. 5,519,063 and European Patent No. EP-A-0 550 745, and more specifically, those described in the polymer preparation Examples 3 and 4 below.

Among the semi-crystalline polymers with a melting point of less than 50° C., non-limiting mention may be made of those described in Examples 3, 4, 5, 7 and 9 of U.S. Pat. No. 5,156,911 with a —COOH group, resulting from the copolymerization of acrylic acid and of C₅ to C₁₆ alkyl(meth)acrylates (with a melting point ranging from 20° C. to 35° C.) and which, for example, may result from the copolymerization:
of acrylic acid, of he xadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of octadecyl methacrylate in a 2.5/97.5 ratio,
of hexadecyl acrylate, of polyethylene glycol monomethyl ether methacrylate with 8 ethylene glycol units and of acrylic acid in an 8.5/1/0.5 ratio by weight.

Use may also be made of the polymer Structure “O” from National Starch, such as that disclosed in U.S. Pat. No. 5,736,125 with a melting point of 44° C., and of semi-crystalline polymers with crystallizable pendent chains comprising fluorinated groups, such as disclosed in Examples 1, 4, 6, 7 and 8 of the publication WO-A-01/19333. Non-limiting mention may also be made of the semi-crystalline polymers of low melting point obtained by copolymerization of stearyl acrylate and of acrylic acid or of NVP as disclosed in U.S. Pat. No. 5,519,063 or European Patent No. EP-A-550 745 and more specifically those described in the polymer preparation Examples 1 and 2 below, with melting points of 40° C. and 38° C. respectively. Further non-limiting mention may be made of the semi-crystalline polymers obtained by copolymerization of behenyl acrylate and of acrylic acid or of NVP as disclosed in U.S. Pat. No. 5,519,063 and European Patent No. EP-A-550 745.

In one embodiment of the present disclosure, the semi-crystalline polymers with a low melting point and/or those with a high melting point do not comprise a carboxyl group.

According to another embodiment of the present disclosure, the polymer results from a monomer with a crystallizable chain chosen from saturated C₁₄ to C₂₂ alkyl (meth)acrylates, for instance from poly(stearyl acrylate)s or poly(behenyl acrylate)s.

The thickening of the fatty phase can be adjusted according to the nature of the polymer or polymers and their concentrations and can be such that a viscosity is obtained, for example, ranging from 1,000 to 250,000 cPs, such as from 10,000 to 50,000 cPs, measured at 25° C. with a Rheomat 180 device with a shear rate of 100 s⁻¹.

The at least one semi-crystalline polymer as defined above can be present for example, in an amount ranging from 0.005% to 20% by weight, for instance ranging from 0.01% to 10% by weight, such as ranging from 0.01% to 1% by weight, with respect to the total weight of the composition.

As used herein, the term “oil” is understood to mean a liquid fatty substance which is insoluble in water at ambient temperature (25° C.) and atmospheric pressure (760 mm of Hg). The oily phase can be composed of at least one oil, and wherein when there is more than one oil, the oils are compatible with one another.

As disclosed herein, the term “insoluble in water” is understood to mean a substance which exhibits a solubility in pure water of less than 1% at 25° C. and at atmospheric pressure.

The oils that can be used as disclosed herein exhibit a dynamic viscosity at 25° C. of less than 1 Pa·s (1000 cPs), for instance, ranging from 10⁻³ to 0.1 Pa·s (from 1 to 100 cPs). The dynamic viscosity is measured at 25° C. with a shear rate of 100 s⁻¹, for example with the device referenced Rheomat RM 180 from Mettler.

Among the oils which can be used as disclosed herein, non-limiting mention may be made, for example, of those chosen from vegetable oils, mineral oils, synthetic oils and fatty acid esters.

Non-limiting mention may be made, for example, among the vegetable oils which can be used as disclosed herein, of sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil and calophyllum oil.

Non-limiting examples of mineral oils that may be used include liquid paraffin and liquid petrolatum.

Among the synthetic oils that can be used, non-limiting mention may be made, for example, of those chosen from polydecenes, squalane, poly(α-olefin)s, such as isododecane or isohexadecane, transesterified vegetable oils and fluorinated oils.

Use may also be made of fatty acid esters, such as, for example, the compounds of formula RₐCOOR_{b} wherein Rₐ is chosen from the residues of a higher fatty acid comprising from 5 to 29 carbon atoms and R_{b} is chosen from hydrocarbonaceous chains comprising from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or 2-octyldodecyl lactate.

For example, the at least one oil may be chosen from avocado oil, castor oil, olive oil, hydrogenated polydecene, isopropyl myristate, isononyl isononanoate and a liquid paraffin.

The at least one oil can be present in the composition as disclosed herein, for example, in an amount ranging from 0.01% to 30% by weight, for instance, in an amount ranging from 0.1% to 15% by weight, such as from 0.1 to 10% by weight, with respect to the total weight of the composition, for example from 0.1% to 5% by weight.

The at least one cationic agent can be chosen from, for example, cationic surfactants and cationic polymers and/or their mixtures. In one embodiment of the present disclosure, the at least one cationic agent is chosen from cationic surfactants.

The cationic polymers which can be used in accordance with the present disclosure can be chosen from all those already known per se as improving the cosmetic properties of the hair, for example, those disclosed in European Patent Application No. EP-A-0 337 354 and in French Patent Application Nos. FR-A-2,270,846, 2,383,660, 2,598,611, 2,470,596 and 2,519,863.

As used herein, the expression “cationic polymer” is understood to mean any polymer comprising cationic groups and/or groups which can be ionized to give cationic groups.

According to one aspect of the present disclosure, the cationic polymers can be chosen from those which comprise units comprising primary, secondary, tertiary and/or quaternary amine groups which can either form part of the main polymer chain or be carried by a side substituent directly connected to the main chain.

The cationic polymers used can have a number- or weight-average molar mass ranging from 500 to 5×10⁶, for instance, ranging from 10³ to 3×10⁶.

Non-limiting mention may be made, for examples among cationic polymers, of the polymers of the polyamine, polyaminoamide and poly(quaternary ammonium) type. These are known products.

Among the polymers of the polyamine, polyaminoamide or poly(quaternary ammonium) type which can be used as disclosed herein non-limiting mention may be made of those disclosed in French Patent Nos. 2,505,348 or 2,542,997. Further non-limiting mention may be made, among these polymers, of:

(1) Homopolymers or copolymers derived from acrylic or methacrylic esters or amides comprising at least one of the units of formulae (III) to (VI): 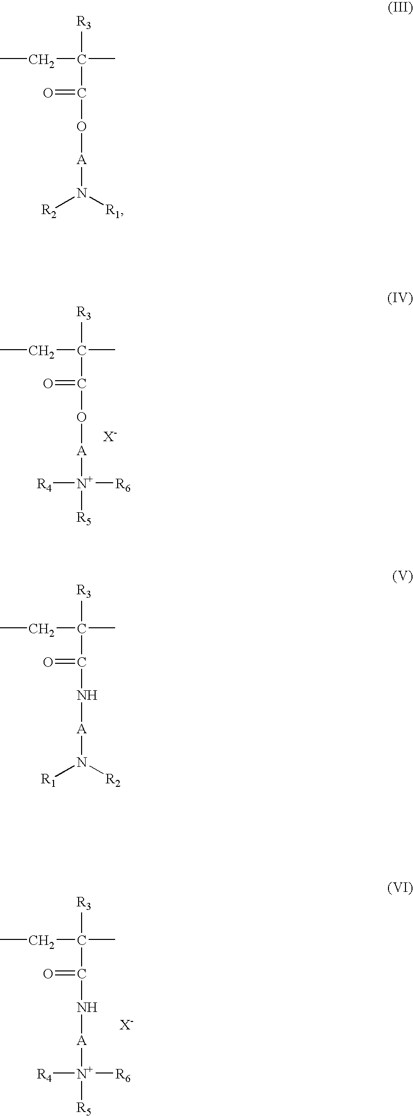
wherein:
R₃, which are identical or different, are chosen from hydrogen atoms and CH₃ radicals;
A, which are identical or different, are chosen from linear and branched alkyl groups comprising from 1 to 6 carbon atoms, such as from 2 to 3 carbon atoms, and hydroxyalkyl groups comprising from 1 to 4 carbon atoms;
R₄, R₅ and R₆, which are identical or different, are chosen from alkyl groups comprising from 1 to 18 carbon atoms and benzyl radicals; for example, R₄, R₅ and R₆ may be chosen from alkyl groups comprising from 1 to 6 carbon atoms;
R₁ and R₂, which are identical or different, are chosen from hydrogen atoms and alkyl groups comprising from 1 to 6 carbon atoms, such as methyl or ethyl;
X is chosen from anions derived from an inorganic or organic acids, such as a methyl sulphate anion, and halides, such as chloride or bromide.

The copolymers of family (1) can additionally comprise at least one unit deriving from comonomers which can be chosen from the family of the acrylamides, methacrylamides, diacetone acrylamides, acrylamides and methacrylamides substituted on the nitrogen by lower (C₁-C₄)alkyls, acrylic or methacrylic acids or their esters, vinyllactams, such as vinylpyrrolidone or vinylcaprolactam, or vinyl esters.

Non-limiting mention may be made, among the copolymers of family (1), of:
copolymers of acrylamide and of dimethylaminoethyl methacrylate which is quaternized with dimethyl sulphate or with a dimethyl halide, such as that sold under the name Hercofloc by Hercules,
copolymers of acrylamide and of methacryloyloxyethyltrimethylammonium chloride, disclosed, for example, in European Patent Application No. EP-A-080 976 and sold under the name Bina Quat P 100 by Ciba-Geigy,
the copolymer of acrylamide and of methacryloyloxyethyltrimethylammonium methyl sulphate sold under the name Reten by Hercules,
vinylpyrrolidone/dialkylaminoalkyl acrylate or methacrylate copolymers, which may or may not be quaternized, such as the products sold under the name “Gafquat” by ISP, like for example “Gafquat 734” or “Gafquat 755”, or the products named “Copolymer 845, 958 and 937.” These polymers are described in detail in French Patent Nos. 2,077,143 and 2,393,573,
dimethylaminoethyl methacrylate/vinylcaprolactam/vinylpyrrolidone terpolymers, such as the product sold under the name Gaffix VC 713 by ISP,
vinylpyrrolidone/methacrylamidopropyldimethylamine copolymers, sold in particular under the name Styleze CC 10 by ISP, and
and vinylpyrrolidone/quaternized dimethylaminopropylmethacrylamide copolymers, such as the product sold under the name “Gafquat HS 100” by ISP.

(2) Cationic polysaccharides, for instance, cationic celluloses and cationic galactomannan gums. Among cationic polysaccharides, non-limiting mention may be made of, for example, cellulose ether derivatives comprising quaternary ammonium groups, cationic cellulose copolymers or cellulose derivatives grafted with a water-soluble quaternary ammonium monomer, and cationic galactomannan gums.

Cellulose ether derivatives comprising quaternary ammonium groups are for example disclosed in French Patent No. 1,492,597 and can be for example, the polymers sold under the names “JR” (JR 400, JR 125, JR 30M) or “LR” (LR 400, LR 30M) by Amerchol. These polymers are also defined in the CTFA dictionary as quaternary ammoniums of hydroxyethylcellulose having reacted with an epoxide substituted by a trimethylammonium group.

Cationic cellulose copolymers or cellulose derivatives grafted with a water-soluble quaternary ammonium monomer are disclosed, for example, in U.S. Pat. No. 4,131,576, such as hydroxyalkyl celluloses, for example hydroxymethyl, hydroxyethyl or hydroxypropyl celluloses, grafted for instance with a methacryloyloxyethyltrimethylammonium, methacrylamidopropyltrimethylammonium or dimethyldiallylammonium salt. The marketed products corresponding to this definition include, for example, the products sold under the name “Celquat L 200” and “Celquat H 100” by National Starch.

Cationic galactomannan gums are disclosed for example, in U.S. Pat. Nos. 3,589,578 and 4,031,307, such as guar gums comprising trialkylammonium cationic groups. Use is made, for example, of guar gums modified by a 2,3-epoxypropyl-trimethylammonium salt (e.g. chloride). Such products are sold for instance under the trade names of Jaguar C13 S, Jaguar C 15, Jaguar C 17 or Jaguar C162 by Rhodia.

(3) Polymers comprised of piperazinyl units and of divalent, straight- or branched-chain alkylene or hydroxyalkylene radicals, optionally interrupted by oxygen, sulphur or nitrogen atoms or by aromatic or heterocyclic rings, as well as the oxidation and/or quaternization products of these polymers. Such polymers are disclosed for example in French Patent Nos. 2,162,025 and 2,280,361.

(4) Water-soluble polyaminoamides prepared for example, by polycondensation of an acidic compound with a polyamine; these polyaminoamides can be crosslinked by an epihalohydrin, a diepoxide, a dianhydride, an unsaturated dianhydride, a bisunsaturated derivative, a bishalohydrin, a bisazetidinium, a bishaloacyldiamine or an alkyl bishalide or alternatively by an oligomer resulting from the reaction of a bifunctional compound reactive with respect to a bishalohydrin, a bisazetidinium, a bishaloacyldiamine, an alkyl bishalide, an epihalohydrin, a diepoxide or a bisunsaturated derivative; the crosslinking agent being used in proportions ranging from 0.025 to 0.35 mol per amine group of the polyaminoamide; these polyaminoamides can be alkylated or, if they comprise at least one tertiary amine functional groups, quaternized. Such polymers are disclosed for instance, in French Patent Nos. 2,252,840 and 2,368,508.

(5) Polyaminoamide derivatives resulting from the condensation of polyalkylenepolyamines with polycarboxylic acids, followed by an alkylation by bifunctional agents. Non-limiting mention may be made, for example, of adipic acid/dialkylaminohydroxyalkyldialkylenetriamine polymers in which the alkyl radical comprises from 1 to 4 carbon atoms such as methyl, ethyl or propyl. Such polymers are disclosed for example, in French Patent No. 1,583,363. Further non-limiting mention may be made, among these derivatives, of the adipic acid/dimethylaminohydroxypropyl/diethylenetriamine polymers sold under the name “Cartaretin F, F4 or F8” by Sandoz.

(6) Polymers obtained by reaction of a polyalkylenepolyamine comprising two primary amine groups and at least one secondary amine group with a dicarboxylic acid chosen from diglycolic acid and saturated aliphatic dicarboxylic acids comprising from 3 to 8 carbon atoms. The molar ratio of polyalkylenepblyamine to dicarboxylic acid ranging from 0.8:1 to 1.4:1; the polyaminoamide resulting therefrom being reacted with epichlorohydrin in a molar ratio of epichlorohydrin in relation to the secondary amine group of the polyaminoamide ranging from 0.5:1 to 1.8:1. Such polymers are disclosed for example, in U.S. Pat. Nos. 3,227,615 and 2,961,347.

Polymers of this type are sold, for instance, under the name “Hercosett 57” by Hercules Inc. or else under the name of “PD 170” or “Delsette 101” by Hercules in the case of the adipic acid/epoxypropyl/diethylenetriamine copolymer.

(7)-Cyclopolymers of alkyldiallylamine or of dialkyldiallylammonium, such as the homopolymers or copolymers comprising, as main constituent of the chain, units of formulae (VII) or (VIII): 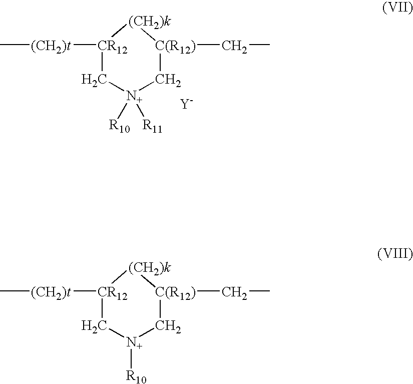
wherein k and t are equal to 0 or 1, the sum k+t being equal to 1; R₁₂ is chosen from a hydrogen atom and methyl radicals; R₁₀ and R₁₁, which can be identical or different, are chosen from alkyl groups comprising from 1 to 6 carbon atoms, hydroxyalkyl groups in which the alkyl group comprises from 1 to 5 carbon atoms, and lower (C₁-C₄)amidoalkyl groups; or, alternatively, R₁₀ and R₁₁ can form, jointly with the nitrogen atom to which they are attached, heterocyclic groups, such as piperidinyl or morpholinyl; Y⁻ is an anion, such as bromide, chloride, acetate, borate, citrate, tartrate, bisulphate, bisulphite, sulphate or phosphate. These polymers are disclosed for instance in French Patent No. 2,080,759 and in its Certificate of Addition 2,190,406.

In one aspect of the present disclosure, R₁₀ and R₁₁, which may be identical or different, are chosen from alkyl groups comprising from 1 to 4 carbon atoms.

Non-limiting mention may be made, for example, among the polymers defined above, of the homopolymer of dimethyldiallylammonium chloride sold under the name “Merquat 100” by Nalco (and its homologues of low weight-average molar masses) and of the copolymers of diallyldimethylammonium chloride and of acrylamide sold under the name “Merquat 550.”

(8) The quaternary diammonium polymers comprising repeat units of formula (IX): 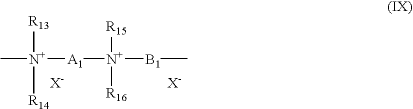
wherein:
R₁₃, R₁₄, R₁₅ and R₁₆, which are identical or different, are chosen from aliphatic, alicyclic and arylaliphatic radicals comprising from 1 to 20 carbon atoms, and lower aliphatic hydroxyalkyl radicals; or, alternatively, R₁₃, R₁₄, R₁₅ and R₁₆, together or separately, form, with the nitrogen atoms to which they are attached, heterocycles optionally comprising a second heteroatom other than nitrogen; or, alternatively, R₁₃, R₁₄, R₁₅ and R₁₆ are chosen from linear and branched C₁-C₆ alkyl radicals substituted by at least one group chosen from nitrile, ester, acyl, amide, —CO—O—R₁₇-D and —CO—NH—R₁₇-D groups, wherein R₁₇ is an alkylene and D a quaternary ammonium group;
   A₁ and B₁ are chosen from linear and branched, saturated and unsaturated polymethylene groups comprising from 2 to 20 carbon atoms, which can optionally comprise, bonded to or inserted into the main chain, at least one entity chosen from aromatic rings, oxygen atoms, sulphur atoms, and sulphoxide, sulphone, disulphide, amino, alkylamino, hydroxyl, quaternary ammonium, ureido, amide and ester groups, and X⁻ is an anion derived from an inorganic or organic acid;
A₁, R₁₃ and R₁₅ can form, with the two nitrogen atoms to which they are attached, a piperazine ring; in addition, if A₁ is chosen from linear and branched, saturated and unsaturated alkylene and hydroxyalkylene radicals, then B₁ can be chosen from —(CH₂)ₙ—CO-D-OC—(CH₂)ₙ— groups wherein D is chosen from:
   a) glycol residues of formula: —O-Z-O—, wherein Z is chosen from linear and branched hydrocarbonaceous radicals and groups of formulae:
      —(CH₂—CH₂—O)ₓ—CH₂—CH₂—
      ——[CH₂—CH(CH₃)—O]_{y}—CH₂—CH(CH₃)—
      wherein x and y are integers ranging from 1 to 4, representing a defined and unique degree of polymerization, or any number from 1 to 4 representing a mean degree of polymerization;
   b) bissecondary diamine residues, such as a piperazine derivative;
   c) bisprimary diamine residues of formula: —NH—Y—NH—, wherein Y is chosen from linear and branched hydrocarbonaceous radicals and the divalent radical —CH₂—CH₂—S—S—CH₂—CH₂—;
   d) ureylene groups of formula: —NH—CO—NH—.
   n, which can be identical or different, is an integer from 2 to 20, such as from 2 to 6,

In one aspect of the present disclosure, X is an anion, such as chloride or bromide.

These polymers have a number-average molar mass ranging, for example, from 1,000 to 100,000.

Polymers of this type are disclosed, for instance, in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

By way of non-limiting example, use may be made, as disclosed herein, of the polymers which are comprised of repeat units of formula (X): 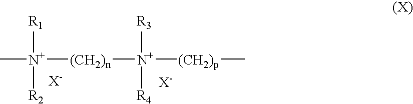
wherein R₁, R₂, R₃ and R₄, which are identical or different, are chosen from alkyl and hydroxyalkyl radicals comprising from 1 to 4 carbon atoms, n and p are integers ranging from 2 to 20, and X⁻ is an anion derived from an inorganic or organic acid.

In one embodiment of the present disclosure, the compound of formula (V) is that for which R₁, R₂, R₃ and R₄ are methyl radicals, n is 3, p is 6 and X is Cl, which is known as Hexadimethrine chloride according to the INCI (CTFA) nomenclature.

(9) Poly(quaternary ammonium)polymers comprising units of formula (XI): 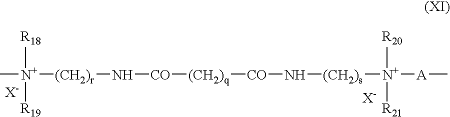
wherein:
R₁₈, R₁₉, R₂₀ and R₂₁, which are identical or different, are chosen from hydrogen atoms, and methyl, ethyl, propyl, β-hydroxyethyl, β-hydroxypropyl and —CH₂CH₂(OCH₂CH₂)ₚOH radicals,
wherein p is an integer ranging from 0 to 6, with the proviso that R₁₈, R₁₉, R₂₀ and R₂₁ are not all simultaneously hydrogen atoms,
r and s, which are identical or different, are integers ranging from 1 to 6,
q is an integer ranging from 0 to 34,
X⁻ is an anion, such as a halide,
A is chosen from radicals of a dihalide, and —CH₂—CH₂—O—CH₂—CH₂— groups.

Such compounds are disclosed for instance in European Patent Application No. EP-A-122 324. Among the polymers of type (9), non-limiting mention may be made, for example, of the products “Mirapol® A 15,” “Mirapol® AD1,” “Mirapol® AZ1” and “Mirapol® 175,” sold by Miranol.

(10) Quaternary polymers of vinylpyrrolidone and of vinylimidazole, such as, for example, the products sold under the names Luviquat® FC 905, FC 550 and FC 370 by BASF.

(11) Polyamines, such as Polyquart® H sold by Cognis, referenced under the name of “Polyethylene Glycol (15) Tallow Polyamine” in the CTFA dictionary.

(12) Polymers, for example, crosslinked polymers of methacryloyloxy(C₁-C₄)alkyltri(C₁-C₄)alkylammonium salts, such as the polymers obtained by homopolymerization of dimethylaminoethyl methacrylate quaternized by methyl chloride or by copolymerization of acrylamide with dimethylaminoethyl methacrylate quaternized by methyl chloride, the homo- or copolymerization being followed by a crosslinking by a compound possessing olefinic unsaturation, such as methylenebisacrylamide. By way of non-limiting example, use may be made of a crosslinked acrylamide/methacryloyloxyethyltrimethylammonium chloride (20/80 by weight) copolymer in the form of a dispersion comprising 50% by weight of the said copolymer in mineral oil. This dispersion is sold under the name of “Salcare® SC 92” by Ciba. As another non-limiting example, use may also be made of a crosslinked homopolymer of methacryloyloxyethyltrimethylammonium chloride comprising 50% by weight of the homopolymer in mineral oil or in a liquid ester. These dispersions are sold under the names of “Salcare® SC 95” and “Salcare® SC 96” by Ciba.

Other cationic polymers that can be used as disclosed herein are cationic proteins or cationic protein hydrolysates, polyalkyleneimines, such as polyethyleneimines, polymers comprising vinylpyridine or vinylpyridinium units, condensates of polyamines and of epichlorohydrin, quaternary polyureylenes and chitin derivatives.

Non-limiting mention may be made, among the cationic polymers mentioned above which are suitable in the present disclosure, for example, of quaternary cellulose ether derivatives, such as the products sold under the name “JR 400” by Amerchol, cationic cyclopolymers, for instance, the homopolymers or copolymers of dimethyldiallylammonium chloride sold under the names “Merquat 100,” “Merquat 550” and “Merquat S” by Nalco, quaternary polymers of vinylpyrrolidone and of vinylimidazole, optionally crosslinked homopolymers or copolymers of methacryloyloxy(C₁-C₄)alkyltri(C₁-C₄)alkylammonium salts, and the mixtures thereof.

The cationic polymer or polymers can be present in an amount ranging from 0.01% to 20%, such as from 0.05% to 10%, for instance from 0.1% to 5% by weight, relative to the total weight of the composition.

The composition according to the present disclosure can also comprise at least one cationic surfactant well known per se, such as salts of optionally polyoxyalkylenated primary, secondary or tertiary fatty amines, quaternary ammonium salts, and their mixtures.

Non-limiting mention may be made, as quaternary ammonium salts, for example, of:
those which exhibit the following general formula (XII): 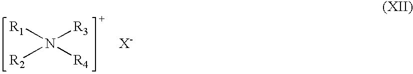
   wherein R₁ to R₄, which can be identical or different, are chosen from linear and branched aliphatic radicals comprising from 1 to 30 carbon atoms, and aromatic radicals, such as aryl or alkylaryl. The aliphatic radicals can comprise heteroatoms, such as, oxygen, nitrogen, sulphur and halogens. The aliphatic radicals are, for example, chosen from alkyl, alkoxy, polyoxy(C₂-C₆)alkylene, alkylamide, (C₁₂-C₂₂)alkylamido(C₂-C₆)alkyl, (C₁₂-C₂₂)alkyl acetate and hydroxyalkyl radicals comprising from 1 to 30 carbon atoms; X⁻ is an anion chosen from halides, phosphates, acetates, lactates, (C₂-C₆)alkyl sulphates, and alkyl- and alkylarylsulphonates,
imidazoline quaternary ammonium salts, such as, for example, those of formula (XIII): 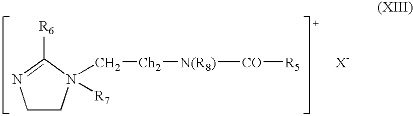
   wherein R₅ is chosen from alkenyl and alkyl radicals comprising from 8 to 30 carbon atoms, for example derivatives of tallow or coconut fatty acids, R₆ is chosen from a hydrogen atom, C₁-C₄ alkyl radicals, and alkenyl and alkyl radicals comprising from 8 to 30 carbon atoms, R₇ is chosen from C₁-C₄ alkyl radicals, R₈ is chosen from a hydrogen atom and C₁-C₄ alkyl radicals, and X⁻ is an anion chosen from halides, phosphates, acetates, lactates, alkyl sulphates, and alkyl- and alkylarylsulphonates. For example, R₅ and R₆ may be a mixture of alkenyl and/or alkyl radicals comprising from 12 to 21 carbon atoms, for example derivatives of tallow fatty acids, R₇ may be a methyl and R₈ may be a hydrogen. Such a product is, for example, Quaternium-27 (CTFA 1997) or Quaternium-83 (CTFA 1997), sold under the names “Rewoquat®” W 75, W90, W75PG and W75HPG” by Witco,
quaternary diammonium salts of formula (XIV): 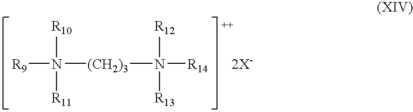
   wherein R₉ is chosen from aliphatic radicals comprising from 16 to 30 carbon atoms, R₁₀, R₁₁, R₁₂, R₁₃ and R₁₄, which are identical or different, are chosen from hydrogen atoms and alkyl radicals comprising from 1 to 4 carbon atoms, and X⁻ is an anion chosen from halides, acetates, phosphates, nitrates, ethyl sulphates-and-methyl sulphates. Such quaternary diammonium salts comprise, for instance, propanetallowdiammonium dichloride,
quaternary ammonium salts comprising at least one ester functional group, such as those of formula (XV): 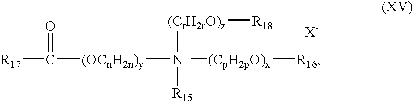
   wherein:
R₁₅ is chosen from linear and branched C₁-C₆ alkyl radicals and C₁-C₆ hydroxyalkyl and dihydroxyalkyl radicals;
R₁₆ is chosen from: 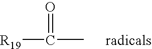
   saturated and unsaturated, linear and branched C₁-C₁₂ hydrocarbonaceous radicals R₂₀, and
   a hydrogen atom,
R₁₈ is chosen from: 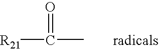
   saturated and unsaturated, linear and branched, C₁-C₆ hydrocarbonaceous radicals R₂₂, and
   a hydrogen atom,
R₁₇, R₁₉ and R₂₁, which are identical or different, are chosen from saturated and unsaturated, linear and branched, C₇-C₂, hydrocarbonaceous radicals;
r, n and p, which are identical or different, are integers ranging from 2 to 6;
y is an integer ranging from 1 to 10;
x and z, which are identical or different, are integers ranging from 0 to 10;
X⁻ is chosen from organic and inorganic, simple and complex anions;
   with the proviso that the sum x+y+z has a value ranging from 1 to 15, that when x is equal to 0, then R₁₆ is the same as R₂₀, and that when z is equal to 0, then R₁₈ is the same as R₂₂.

According to one aspect of the present disclosure, for instance, R₁₅ may be chosen from methyl, ethyl, hydroxyethyl and dihydroxypropyl radicals, such as methyl and ethyl radicals. In another aspect of the present disclosure, for example, the sum x+y+z ranges from 1 to 10.

When R₁₆ is an R₂₀ hydrocarbonaceous radical, it can be long and comprise from 12 to 22 carbon atoms, or short and comprise from 1 to 3 carbon atoms.

When R₁₈ is an R₂₂ hydrocarbonaceous radical, it may comprise, for example, 1 to 3 carbon atoms.

R₁₇, R₁₉ and R₂₁, which are identical or different, may be chosen from, for instance, saturated and unsaturated, linear and branched, C₁₁-C₂₁ hydrocarbonaceous radicals, such as from saturated and unsaturated, linear and branched, C₁₁-C₂₁ alkyl and alkenyl radicals. In one embodiment of the present disclosure, for example, x and z, which are identical or different, are equal to 0 or 1.

In another embodiment of the present disclosure, y is equal to 1.

For example, r, n, and p, which are identical or different, may have a value of 2 or 3 and, in one embodiment, are equal to 2.

The anion X⁻ may be chosen from, for example, a halides (chloride, bromide or iodide) and (C₁-C₄)alkyl sulphates, such as methyl sulphate. However, it is also possible to use methanesulphonate, phosphate, nitrate, tosylate, an anion derived from an organic acid, such as acetate or lactate, or any other anion compatible with ammonium comprising an ester functional group.

In one embodiment of the present disclosure, the X⁻ anion is chosen from chloride and methyl sulphate.

As disclosed herein, by way of non-limiting example, use may be made, of the ammonium salts of formula (IX) wherein:
R₁₅ may be chosen from methyl and ethyl radicals,
x and y are equal to 1;
z is equal to 0 or 1;
r, n and p are equal to 2;
R₁₆ may be chosen from: 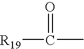
radicals; methyl, ethyl and C₁₄-C₂₂ hydrocarbonaceous radicals; and a hydrogen atom;
R₁₈ may be chosen from: 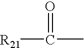
radicals, and a hydrogen atom; and
R₁₈, R₁₉ and R₂₁, which are identical or different, may be chosen from saturated and unsaturated, linear and branched, C₁₃-C₁₇ hydrocarbonaceous radicals and for instance, from saturated and unsaturated, linear and branched, C₁₃-C₁₇ alkyl and alkenyl radicals. In one aspect of the present disclosure, the hydrocarbonaceous radicals are linear.

Non-limiting mention may be made, for example, of the compounds of formula (XV), such as diacyloxyethyldimethylammonium, diacyloxyethyl(hydroxyethyl)methylammonium, monoacyloxyethyl(dihydroxyethyl)methylammonium, triacyloxyethyl(methyl)ammonium or monoacyloxyethyl(hydroxyethyl)dimethylammonium salts (such as chloride or methyl sulphate) and their mixtures. The acyl radicals may have, for example, 14 to 18 carbon atoms and, for instance, may originate from a vegetable oil, such as palm oil or sunflower oil. When the compound comprises several acyl radicals, the latter can be identical or different.

These products are obtained, for example, by direct esterification of triethanolamine, of triisopropanolamine, of alkyldiethanolamine or of alkyldiisopropanolamine, which are optionally oxyalkylenated, with fatty acids or with mixtures of fatty acids of vegetable or animal origin or by transesterification of their methyl esters. This esterification is followed by a quaternization using an alkylating agent, such as an alkyl (such as methyl or ethyl) halide, a dialkyl (such as methyl or ethyl) sulphate, methyl methanesulphonate, methyl para-toluenesulphonate, or glycol or glycerol chlorohydrin.

Such compounds are, for example, sold under the names Dehyquart by Cognis, Stepanqua® by Stepan, Noxamium® by Ceca or Rewoqua® WE 18 by Rewo-Goldschmidt.

The composition according to the present disclosure can comprise for example, a mixture of quaternary ammonium mono-, di- and triester salts, with a majority by weight of diester salts.

Use may be made, as mixture of ammonium salts, of, for example, the mixture comprising 15 to 30% by weight of acyloxyethyl(dihydroxyethyl)methylammonium methyl sulphate, 45 to 60% of diacyloxyethyl(hydroxyethyl)methylammonium methyl sulphate and 15 to 30% of triacyloxyethyl(methyl)ammonium methyl sulphate, the acyl radicals comprising from 14 to 18 carbon atoms and originating from optionally partially hydrogenated palm oil.

It is also possible to use the ammonium salts comprising at least one ester functional group disclosed in U.S. Pat. Nos. 4,874,554 and 4,137,180.

Non-limiting mention may be made, among the abovementioned quaternary ammonium salts, of the use of those corresponding to the formula (XII). Further non-limiting mention may for example, be made of, on the one hand, tetraalkylammonium chlorides, such as, for example, dialkyldimethylammonium or alkyltrimethylammonium chlorides, in which the alkyl radical comprises from 12 to 22 carbon atoms, such as behenyltrimethylammonium, distearyldimethylammonium, cetyltrimethylammonium and benzyldimethylstearyl-ammonium chlorides, or alternatively, on the other hand, of palmitylamidopropyltrimethyl ammonium chloride or stearylamidopropyldimethyl(myristyl acetate)ammonium chloride, sold under the name Ceraphyl® 70 by Van Dyk.

In one aspect of the present disclosure, the cationic surfactants may be chosen from, for example, quaternary ammonium salts, such as behenyltrimethylammonium chloride, cetyltrimethylammonium chloride, quaternium-83, behenylamidopropyl(2,3-dihydroxypropyl)dimethylammonium chloride and palmitylamidopropyltrimethylammonium chloride.

The composition according to the present disclosure comprises the at least one cationic agent in an amount ranging from 0.05% to 10% by weight, for instance, from 0.1% to 8% by weight, relative to the total weight of the composition, such as from 0.2% to 6% by weight, for example from 0.3% to 3% by weight.

The composition according to the present disclosure can optionally comprise at least one surfactant other than a cationic surfactant, such as nonionic or amphoteric surfactants.

The at least one additional surfactant can be present in an amount ranging from 0.1% to 10% by weight, such as ranging from 0.5% to 8%, for example ranging from 1% and 5%, relative to the total weight of the composition.

In one embodiment of the present disclosure, the composition comprises at least one surfactant chosen from nonionic surfactants.

The nonionic surface-active agents themselves are also compounds well known per se (in this respect, see for example, the Handbook of Surfactants, by M. R. Porter, published by Blackie & Son (Glasgow and London), 1991, pp. 116-178) and, in the context of the present disclosure, their nature does not take on a critical character. Thus, among the nonionic surfactants that may be used, non-limiting mention may be made of: polyethoxylated, polypropoxylated or polyglycerolated fatty alcohols, polyethoxylated, polypropoxylated or polyglyerolated fatty α-diols, polyethoxylated, polypropoxylated or polyglycerolated fatty alkylphenols or polyethoxylated, polypropoxylated or polyglycerolated fatty acids, all these compounds having a fatty chain comprising, for example, 8 to 18 carbon atoms, it being possible for the number of ethylene oxide or propylene oxide groups to range from 2 to 50 and it being possible for the number of glycerol groups to range from 2 to 30. Non-limiting mention may also be made of copolymers of ethylene oxide and of propylene oxide, condensates of ethylene oxide and of propylene oxide with fatty alcohols; polyethoxylated fatty amides comprising from 2 to 30 mol of ethylene oxide, polyglycerolated fatty amides comprising from 1 to 5 glycerol groups, such as from 1.5 to 4; sorbitan oxyethylenated fatty acid esters comprising from 2 to 30 mol of ethylene oxide; sucrose fatty acid esters, polyethylene glycol esters of fatty acids, alkylpolyglycosides, N-alkylglucamine derivatives or amine oxides, such as oxides of (C₁₀-C₁₄)alkylamines or N-acylaminopropylmorpholine oxides. In one embodiment of the present disclosure, the nonionic surfactants are chosen from alkylpolyglycosides.

The nonwashing (nondetergent) compositions comprise, for example, less than 4% by weight of anionic detergent surfactants, such as less than 1% by weight, relative to the total weight of the composition.

The composition according to the present disclosure can additionally comprise at least one conditioning agent.

The at least one conditioning agent, such as cationic polymers, can be present in the composition as disclosed herein in an amount ranging from 0.01% to 20% by weight, for instance, ranging from 0.05% to 10% by weight and such as ranging from 0.1% to 5% by weight, relative to the total weight of the composition.

The cosmetically acceptable medium can be, for example, aqueous and can comprise water or a mixture of water and at least one cosmetically acceptable solvent chosen from lower C₁-C₄ alcohols, for example ethanol, isopropanol, tert-butanol or n-butanol; polyols, such as propylene glycol or glycerol; polyol ethers; and C₅-C₁₀ alkanes. For example, in one aspect of the present disclosure, the at least one cosmetically acceptable solvent is chosen from glycerol and propylene glycol.

The cosmetically acceptable, for instance, aqueous, medium can be present in the composition in an amount ranging from 30% to 98% by weight, relative to the total weight of the composition, such as from 80% to 98% by weight.

The at least one cosmetically acceptable solvent, when present, can be present in the composition in an amount ranging from 0.5% to 30% by weight, with respect to the total weight of the composition.

The pH of the compositions as disclosed herein ranges from 2 to 8, for instance, from 3 to 7.5.

The compositions according to the present disclosure can also comprise at least one conventional additive well known in the art, such as anionic, nonionic or amphoteric polymers, nonpolymeric thickeners, such as acids or electrolytes, opacifying agents, pearlescence agents, vitamins, provitamins, such as panthenol, fatty alcohols, waxes, such as vegetable waxes, natural or synthetic ceramides, fragrances, colorants, organic or inorganic particles, preservatives or pH stabilizing agents.

A person skilled in the art will take care to choose the optional additives and their amounts so that they do not harm the properties of the compositions of the present invention.

The at least one additive can be present in the composition as disclosed herein in an amount ranging from 0% to 20% by weight, relative to the total weight of the composition.

The compositions of the invention can be provided in the form of a rinse-out or leave-in conditioner, of compositions for perming, hair straightening, dyeing or bleaching, or in the form of compositions, optionally to be rinsed out, to be applied before or after a dyeing, a bleaching, a perming or a hair straightening or alternatively between the two stages of a perming or of a hair straightening.

They can be used, for example, as conditioners, care products, deep care masks, or lotions or creams for treating the scalp. These compositions can be rinse-out or leave-in compositions.

For example, according to one embodiment of the invention, the composition can be used as a conditioner, for instance for fine hair. This conditioner can be a rinse-out or leave-in conditioner, and in another embodiment is a rinse-out conditioner.

The cosmetic compositions according to the present disclosure can be provided in the form of a gel, of a milk, of a cream, of an emulsion, of a fluid or thickened lotion or of a foam and can be used for the skin, the nails, the eyelashes, the lips and, for example, the hair.

The compositions can be packaged in various forms, for instance, in vaporizers, pump-action sprays or aerosol containers, in order to provide for application of the composition in the vaporized form or in a foam form. Such packaging forms are indicated, for example, when it is desired to obtain a spray, a lacquer or a foam for hair treatment.

The present disclosure also relates to a process for the cosmetic treatment of keratinous substances, such as, for example, the skin or the hair, which comprises applying an effective amount of a cosmetic composition as described above to the keratinous substances, after an optional leave-in time, in optionally rinsing. Rinsing is carried out, for example, with water.

Thus, the process as disclosed herein can make possible form retention of the hairstyle and the treatment, conditioning or care of the hair or any other keratinous substance.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting manner.

### EXAMPLES 1 and 2

The following rinse-out conditioning compositions were prepared:

| | Composition | |
|---|---|---|
| | 1 | 2 |
| Inulin (Raftiline HP, Orafti) | 15 g | — |
| Polyquaternium-37 | 0.5 g | — |
| (Salcare SC 95, Ciba) | | |
| Cetearyl alcohol | — | 2 g |
| Cetyl esters (Spermwax vegetal, | — | 0.25 g |
| Robeco) | | |
| Behentrimonium chloride | — | 1.4 g |
| (Genamin KDMP, Clariant) | | |
| Mixture (6/94 by weight) | 0.5 g | 1 g |
| of poly (C₁₀-C₃₀ alkyl | | |
| acrylate) (Intelimer IPA13-1 | | |
| from Landec) and of Hydrogenated | | |
| polydecene | | |
| (Ceraflow E from Shamrock) | | |
| Preservative | q.s. | q.s. |
| pH agent q.s. | pH = 7 | pH = 3.5 |
| Water | q.s. for 100 g | q.s. for 100 g |

Compositions 1 and 2 were applied separately to different locks of sensitized hair. After leaving in for approximately three minutes, the hair was rinsed. This hair was then very smooth.

### EXAMPLES 3-4

The following rinse-out conditioning compositions were prepared:

| | Composition | |
|---|---|---|
| | 3 | 4 |
| Inulin (Raftiline HP, Orafti) | 15 g | — |
| Polyquaternium-37 | 0.5 g | — |
| (Salcare SC 95, Ciba) | | |
| Cetearyl alcohol | — | 2 g |
| Cetyl esters (Spermwax vegetal, | — | 0.25 g |
| Robeco) | | |
| Behentrimonium chloride (Genamin | — | 1.4 g |
| KDMP, Clariant) | | |
| Mixture (6/94 by weight) | 0.5 g | 1 g |
| of poly(C₁₀-C₃₀ alkyl | | |
| acrylate) (Intelimer IPA13-1 | | |
| from Landec) and of avocado oil | | |
| Preservative | q.s. | q.s. |
| pH agent q.s. | pH = 7 | pH = 3.5 |
| Water | q.s. for 100 g | q.s. for 100 g |

Compositions 3 and 4 were applied separately to different locks of sensitized hair. After leaving in for approximately three minutes, the hair was rinsed. The hair was then very smooth.

### EXAMPLE 5-6

The following rinse-out conditioning compositions were prepared:

| | Composition | |
|---|---|---|
| | 5 | 6 |
| Inulin (Raftiline HP, Orafti) | 15 g | — |
| Polyquaternium-37 | 0.5 g | — |
| (Salcare SC 95, Ciba) | | |
| Cetearyl alcohol | — | 2 g |
| Cetyl esters (Spermwax vegetal, | — | 0.25 g |
| Robeco) | | |
| Behentrimonium chloride (Genamin | — | 1.4 g |
| KDMP, Clariant) | | |
| Mixture (15/85 by weight) of C₁₀-C₃₀ | 0.5 g | 1 g |
| alkyl acrylate/methacrylic acid | | |
| copolymer and of isononyl | | |
| isononanoate | | |
| Preservative | q.s. | q.s. |
| pH agent q.s. | pH = 7 | pH = 3.5 |
| Water | q.s. for 100 g | q.s. for 100 g |

Compositions 5 and 6 were applied separately to different locks of sensitized hair. After leaving in for approximately three minutes, the hair was rinsed. The hair was then very smooth.

1. A cosmetic composition comprising, in a cosmetically acceptable medium, at least one cationic agent, at least one oil, and at least one semi-crystalline polymer having a melting point of greater than or equal to 30° C.

2. The composition according to claim 1, wherein the at least one semi-crystalline polymer has a melting point ranging from 30° C. to 80° C.

3. The composition according to claim 2, wherein the at least one semi-crystalline polymer has a melting point ranging from 30° C. to 70° C.

4. The composition according to claim 1, the at least one semi-crystalline polymer has a weight-average molecular mass of greater than or equal to 1,000.

5. The composition according to claim 1, wherein the at least one semi-crystalline polymer has a number-average molecular mass ranging from 5,000 to 1,000,000.

6. The composition according to claim 5, wherein the at least one semi-crystalline polymer has a number-average molecular mass ranging from 10,000 to 500,000.

7. The composition according to claim 1, wherein the at least one semi-crystalline polymer comprises
a polymer backbone, and
at least one crystallizable side chain and/or at least one crystallizable organic block forming part of the backbone of the at least one semi-crystalline polymer.

8. The composition according to claim 1, wherein the at least one semi-crystalline polymer is chosen from block copolymers comprising at least one crystallizable block and at least one amorphous block; and homopolymers and copolymers carrying at least one crystallizable side chain per repeating unit.

9. The composition according to claim 8, wherein the crystallizable block is different in nature from the amorphous block.

10. The composition according to claim 8, wherein the at least one semi-crystalline polymer comprises a crystallizable organic chain and/or a crystallizable block that is present in an amount equal to or greater than 30% by weight, relative to the total weight of the polymer.

11. The composition according to claim 10, wherein the crystallizable organic chain and/or crystallizable block is present in an amount equal to or greater than 40% by weight, relative to the total weight of the polymer.

12. The composition according to claim 1, wherein the at least one semi-crystalline polymer is chosen from:
block copolymers of polyolefins with controlled crystallization,
aliphatic and aromatic polyester polycondensates and aliphatic/aromatic copolyesters, and
homo- and copolymers carrying at least one crystallizable side chain.

13. The composition according to claim 7, wherein the at least one semi-crystalline polymer is chosen from homopolymers and copolymers comprising from 50% to 100% by weight of units resulting from the polymerization of at least one monomer carrying at least one crystallizable hydrophobic side chain.

14. The composition according to claim 1, wherein the at least one semi-crystalline polymer is chosen from homopolymers and block copolymers resulting from the polymerization of at least one monomer with at least one crystallizable side chain per repeating unit of formula (I): 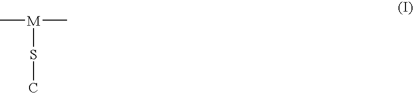
wherein M is an atom of the polymer backbone, S is a spacer, and C is chosen from crystallizable groups, and further wherein “S—C” is an optionally fluorinated or perfluorinated alkyl chain comprising from 11 to 40 carbon atoms.

15. The composition according to claim 14, wherein the at least one semi-crystalline polymer is chosen from the polymers resulting from the polymerization of at least one monomer chosen from acrylic acid, methacrylic acid, crotonic acid, itaconic acid, maleic acid, and maleic anhydride.

16. The composition according to claim 1, wherein the at least one semi-crystalline polymer is chosen from the homopolymers and copolymers resulting from the polymerization of at least one monomer with a crystallizable chain chosen from saturated C₁₄-C₂₄ alkyl(meth)acrylates; C₁₁-C₁₅ perfluoroalkyl(meth)acrylates; N-(C₁₄ to C₂₄ alkyl)-(meth)acrylamides, with or without at least one fluorine atom; vinyl esters with C₁₄ to C₂₄ alkyl chains; vinyl esters with C₁₄ to C₂₄ perfluoroalkyl chains; vinyl ethers with C₁₄ to C₂₄ alkyl chains; vinyl ethers with C₁₄ to C₂₄ perfluoroalkyl chains; C₁₄ to C₂₄ α-olefins; and para-alkylstyrenes with an alkyl group comprising from 12 to 24 carbon atoms.

17. The composition according to claim 16, wherein the at least one semi-crystalline polymer is chosen from the copolymers resulting from the polymerization of at least one monomer with a crystallizable chain, chosen from saturated C₁₄ to C₂₄ alkyl (meth)acrylates; C₁₁ to C₁₅ perfluoroalkyl(meth)acrylates; N-(C₁₄ to C₂₄ alkyl)methacrylamides, with or without at least one fluorine atom; vinyl esters with C₁₄ to C₂₄ alkyl chains; vinyl esters with C₁₄ to C₂₄ perfluoroalkyl chains; vinyl ethers with C₁₄ to C₂₄ alkyl chains; vinyl ethers with C₁₄ to C₂₄ perfluoroalkyl chains; C₁₄ to C₂₄ α-olefins; and para-alkylstyrenes with an alkyl group comprising from 12 to 24 carbon atoms, with at least one optionally fluorinated C₁ to C₁₀ monocarboxylic acid ester or amide.

18. The composition according to claim 17, wherein the at least one semi-crystalline polymer is chosen from homopolymers of alkyl(meth)acrylate with a C₁₄ to C₂₄ alkyl group, alkyl(meth)acrylamide with a C₁₄ to C₂₄ alkyl group, and the copolymers of these monomers with a hydrophilic monomer.

19. The composition according to claim 18, wherein the at least one semi-crystalline polymer is a copolymer of alkyl(meth)acrylate with a C₁₄ to C₂₄ alkyl group or of alkyl(meth)acrylamide with a C₁₄ to C₂₄ alkyl group, with a hydrophilic monomer which is different in nature from (meth)acrylic acid, and the mixtures thereof.

20. The composition according to claim 19, wherein the hydrophilic monomer which is different in nature from (meth)acrylic acid is chosen from N-vinylpyrrolidone and hydroxyethyl(meth)acrylate.

21. The composition according to claim 18, wherein the at least one semi-crystalline polymer results from a monomer with a crystallizable chain chosen from saturated C₁₄ to C₂₂ alkyl(meth)acrylates.

22. The composition according to claim 21, wherein the at least one semi-crystalline polymer is chosen from poly(stearyl acrylate)s or poly(behenyl acrylate)s.

23. The composition according to claim 1, wherein the at least one semi-crystalline polymer is present in the composition in an amount ranging from 0.01% to 10% by weight, relative to the total weight of the composition.

24. The composition according to claim 23, wherein the at least one semi-crystalline polymer is present in the composition in an amount ranging from 0.05% to 5% by weight, relative to the total weight of the composition.

25. The composition according to claim 1, wherein the at least one oil is chosen from vegetable oils, mineral oils, synthetic oils and fatty acid esters.

26. The composition according to claim 25, wherein the at least one oil is chosen from sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil, calophyllum oil, liquid paraffin, liquid petrolatum, polydecenes, squalane, poly(α-olefins), transesterified vegetable oils and fluorinated oils, and compounds of formula RₐCOOR_{b} wherein Rₐ is the residue of a higher fatty acid comprising from 5 to 29 carbon atoms and R_{b} is a hydrocarbonaceous chain comprising from 3 to 30 carbon atoms.

27. The composition according to claim 26, wherein the poly(α-olefins) are chosen from isododecane and isohexadecane.

28. The composition according to claim 26, the at least one oil is chosen from avocado oil, castor oil, olive oil, isohexadecane, polydecene, isopropyl myristate, isononyl isononanoate and liquid paraffin.

29. The composition according to claim 1, wherein the at least one oil is present in an amount ranging from 0.01% to 30% by weight, relative to the total weight of the composition.

30. The composition according to claim 29, wherein the at least one oil is present in an amount ranging from 0.1% to 15% by weight, relative to the total weight of the composition.

31. The composition according to claim 1, wherein the at least one oil is pre-thickened by the at least one semi-crystalline polymer.

32. The composition according to claim 1, wherein the weight ratio of the at least one oil to the at least one semi-crystalline polymer is greater than or equal to 50/50.

33. The composition according to claim 32, wherein the weight ratio of the at least one oil to the at least one semi-crystalline polymer is greater than or equal to 60/40.

34. The composition according to claim 33, wherein the weight ratio of the at least one oil to the at least one semi-crystalline polymer ranges from 60/40 to 99/1.

35. The composition according to claim 1, wherein the at least one oil exhibits a number-average primary size ranging from 1 μm to 100 μm.

36. The composition according to claim 35, wherein the at least one oil exhibits a number-average primary size ranging from 5 μm to 30 μm.

37. The composition according to claim 1, wherein the at least one cationic agent is chosen from cationic polymers and cationic surfactants.

38. The composition according to claim 37, wherein the cationic polymers are chosen from quaternary cellulose ether derivatives, cationic cyclopolymers, quaternary polymers of vinylpyrrolidone and of vinylimidazole, optionally crosslinked homopolymers and copolymers of methacryloyloxy(C₁-C₄)alkyltri(C₁-C₄)alkylammonium salts, and mixtures thereof.

39. The composition according to claim 38, wherein the cationic cyclopolymers, are chosen from homopolymers and copolymers of dimethyldiallylammonium chloride.

40. The composition according to claim 37, wherein the cationic surfactants are chosen from salts of optionally polyoxyalkylenated primary, secondary and tertiary fatty amines, quaternary ammonium salts, and mixtures thereof.

41. The composition according to claim 40, wherein the quaternary ammonium salts are chosen from:
those of formula (XII): 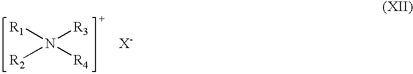
wherein R₁ to R₄, which can be identical or different, are chosen from linear and branched aliphatic radicals comprising from 1 to 30 carbon atoms, and aromatic radicals; X⁻ is an anion chosen from halides, phosphates, acetates, lactates, (C₂-C₆)alkyl sulphates, and alkyl- and alkylarylsulphonates,
imidazoline quaternary ammonium salts
quaternary diammonium salts of formula (XIV): 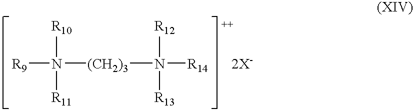
wherein R₉ is chosen from aliphatic radicals comprising from 16 to 30 carbon atoms, R₁₀, R₁₁, R₁₂, R₁₃ and R₁₄, which are identical or different, are chosen from hydrogen atoms, and alkyl radicals comprising from 1 to 4 carbon atoms; and X⁻ is an anion chosen from halides, acetates, phosphates, nitrates and methyl sulphates; and
quaternary ammonium salts comprising at least one ester functional group.

42. The composition according to claim 41, wherein the aromatic radicals are chosen from aryl and alkylaryl radicals.

43. The composition according to claim 41, wherein the imidazoline quaternary ammonium salts are chosen from those of formula (XIII): 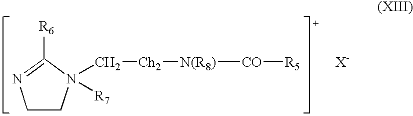
wherein R₅ is chosen from alkenyl and alkyl radicals comprising from 8 to 30 carbon atoms; R₆ is chosen from a hydrogen atom, C₁-C₄ alkyl radicals and alkenyl and alkyl radicals comprising from 8 to 30 carbon atoms; R₇ is chosen from C₁-C₄ alkyl radicals; R₈ is chosen from a hydrogen atom and C₁-C₄ alkyl radicals; and X⁻ is an anion chosen from halides, phosphates, acetates, lactates, alkyl sulphates, and alkyl- and alkylarylsulphonates.

44. The composition according to claim 43, wherein R₅ and R₆ are chosen from a mixture of alkenyl and alkyl radicals comprising from 12 to 21 carbon atoms; R₇ is a methyl and R₈ is a hydrogen.

45. The composition according to claim 44, wherein R₅ and R₆ are chosen from derivatives of tallow fatty acids or coprah fatty acids.

46. The composition according to claim 40, wherein the cationic surfactants are chosen from behenyltrimethylammonium chloride, cetyltrimethylammonium chloride, Quaternium-83, behenylamidopropyl(2,3-dihydroxypropyl)dimethylammonium chloride and palmitylaminopropyltrimethylammonium chloride.

47. The composition according claim 37, wherein the at least one cationic surfactant is present in an amount ranging from 0.05% to 10% by weight, relative to the total weight of the composition.

48. The composition according claim 47, wherein the at least one cationic surfactant is present in an amount ranging from 0.1% to 8% by weight, relative to the total weight of the composition.

49. The composition according to claim 1, further comprising at least one additional conditioning agent.

50. The composition according to claim 1, wherein the cosmetically acceptable aqueous medium comprises water or of a mixture of water and at least one cosmetically acceptable solvent.

51. The composition according to claim 50, wherein the at least one cosmetically acceptable solvent is chosen from lower C₁-C₄ alcohols; alkylene glycols; polyol ethers; and C₅-C₁₀ alkanes.

52. The composition according to claim 51, wherein the lower C₁-C₄ alcohols are chosen from ethanol, isopropanol, tert-butanol, and n-butanol.

53. The composition according to claim 52, wherein the alkylene glycols are propylene glycol.

54. The composition according to claim 1, further comprising at least one additive chosen from anionic, nonionic and amphoteric polymers; thickeners; opacifying agents; pearlescence agents; vitamins; provitamins; waxes; natural and synthetic ceramides; fragrances; colorants; organic and inorganic particles; preservatives; and pH stabilizing agents.

55. The composition according to claim 54, wherein the thickeners are acids and electrolytes.

56. The composition according to claim 54, wherein the provitamin is panthenol.

57. The composition according to claim 54, wherein the waxes are chosen from vegetable waxes.

58. The composition according to claim 1, wherein it is provided in a form chosen from: a rinse-out conditioner, a leave-in conditioner, a composition for perming the hair, a composition for straightening the hair, a composition for dyeing the hair, a composition for bleaching the hair, a rinse-out composition to be applied between the two stages of a perming, a rinse-out composition to be applied between the two stages of a hair straightening, and a composition to be applied before and/or after perming, straightening, dyeing and/or bleaching the hair.

59. A process for the treatment of keratinous substances, comprising applying, to the keratinous substances, an effective amount of a cosmetic composition comprising, in a cosmetically acceptable medium, at least one cationic agent, at least one oil and at least one semi-crystalline polymer having a melting point of greater than or equal to 30° C.,
and then optionally rinsing the keratinous substances.

60. The process according to claim 59, wherein the keratinous substances are the hair.
